(19) United States
(12) Patent Application Publication
(10) Pub. No.: US 2024/0050668 A1
(43) Pub. Date: Feb. 15, 2024

(54) DEVICES, ASSEMBLIES, AND METHODS FOR DELIVERING POWDERED AGENTS

(71) Applicant: Boston Scientific Scimed, Inc., Maple Grove, MN (US)

(72) Inventor: Henry STOCK, Sanbornton, NH (US)

(73) Assignee: Boston Scientific Scimed, Inc., Maple Grove, MN (US)

(21) Appl. No.: 18/449,056

(22) Filed: Aug. 14, 2023

Related U.S. Application Data
(60) Provisional application No. 63/371,437, filed on Aug. 15, 2022.

(51) Int. Cl. A61M 13/00

(52) U.S. Cl. CPC A61M 13/00; A61M 2202/064; A61M 2205/3341; A61M 2205/123

(57) ABSTRACT

A pressure relief mechanism that includes a first housing including a first body between a first and second wall, a cavity within the first body configured to receive a pressure relief device, a first lip extending from the first wall, and a second lip extending from the second wall. The pressure relief mechanism includes a second housing including a second body between a third and fourth wall, a first projection extending from the third wall, and a second projection extending from the fourth wall. The first projection engages the first lip and the second projection engages the second lip to couple the first and second housing with the pressure relief device enclosed within the cavity. The first and second housing are configured to apply a clamping force along a perimeter of the pressure relief device to seal the pressure relief device between the first and second housing.

### CROSS-REFERENCE TO RELATED APPLICATIONS

This application claims the benefit of priority of U.S. Provisional Patent Application No. 63/371,437, filed on Aug. 15, 2022, the entirety of which is incorporated herein by reference.

### TECHNICAL FIELD

Various aspects of this disclosure relate generally to devices for delivering agents. More specifically, in embodiments, this disclosure relates to pressure relief mechanisms incorporated into devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a device inserted into the working channel of the endoscope. Agent delivery may be achieved, for example, through the use of pressurized fluid that move the agent through the device and toward the remote site. Such systems, however, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract when the pressurized fluid exceeds a desired pressurization level. Pressure relief mechanisms may ensure the pressurized fluid is delivered at the desired pressurization level, but a housing of the mechanism may be complex, large, or require numerous tools or steps for assembly. The current disclosure may solve one or more of these issues or other issues in the art.

### SUMMARY

Each of the aspects disclosed herein may include one or more of the features described in connection with any of the other disclosed aspects.

Aspects of the disclosure relate to, among other things, systems, devices, and methods relating to pressure relief mechanisms incorporated into dispensing devices used to deliver various agents (e.g., powdered agents) to treatment sites within patients during certain medical procedures. According to an example, a pressure relief mechanism may include a first housing including a first body defined between a first wall and a second wall that is opposite of the first wall, a cavity disposed within the first body between the first wall and the second wall, the first body being configured to receive a pressure relief device within the cavity, a first lip extending outward from the first wall; and a second lip extending outward from the second wall, and a second housing including a second body defined between a third wall and a fourth wall that is opposite of the third wall, a first projection extending outwardly from the third wall, and a second projection extending outwardly from the fourth wall, where the first projection is configured to engage the first lip and the second projection is configured to engage the second lip to couple the first housing to the second housing with the pressure relief device enclosed within the cavity, where the first housing and the second housing are configured to apply a clamping force along a perimeter of the pressure relief device to fluidly seal the pressure relief device between the first housing and the second housing.

Any of the pressure relief mechanisms described herein may include any of the following features. The first housing includes an input port disposed through a front wall of the first body, and an output port disposed through a rear wall of the first body, wherein each of the input port and the output port are in fluid communication with the cavity. The first housing is configured to receive a pressurized fluid within the cavity via the input port, and release the pressurized fluid from within the cavity via the output port. The first projection includes a tapered clip configured to form a snap fit connection with the first lip such that the second housing is immovable relative to the first housing when the tapered clip is coupled to the first lip. The tapered clip is elastically deformable, and configured to bend laterally outward relative to the third wall as the second housing is coupled to the first housing to form the snap fit connection. The tapered clip of the first projection includes a hook that extends from the third wall towards the fourth wall, and wherein the hook is configured to engage a lower interface of the first lip. The second projection includes a rounded clip configured to form a hinged connection with the second lip, such that the second housing is movable relative to the first housing about the hinged connection when the rounded clip is coupled to the second lip. The rounded clip of the second projection includes a ball joint that extends from the fourth wall towards the third wall, and wherein the ball joint is configured to pivot about an axis when received along a lower interface of the second lip. The second housing is configured to move between a first position and a second position relative to the first housing about the axis. In the first position, the first projection is disengaged from the lower interface of the first lip, and in the second position, the first projection is engaged with the lower interface of the first lip. The first housing includes at least one recessed surface along the second wall, the at least one recessed surface forming a first interior wall and a second interior wall positioned opposite of the first interior wall. The second projection of the second housing has a width that corresponds to a distance between the first interior wall and the second interior wall, such that the second projection is configured to fit between the first interior wall and the second interior wall when the second projection engages the second lip. The first interior wall and the second interior wall are configured to inhibit lateral movement of the second projection relative to the second lip when the first housing is coupled to the second housing. The second housing includes an opening extending through the second body, and is aligned with the cavity when the first housing is coupled to the second housing. The opening is configured to release a pressurized fluid received in the cavity of the first housing upon the pressure relief device rupturing.

According to another example, a pressure relief mechanism may include a first housing including a first body, a cavity disposed within the first body and configured to receive a pressure relief device, a first lip extending outward from a first wall of the first body, and a second lip extending outward from a second wall of the first body, and a second housing including a second body, a first projection extending outward from a first wall of the second body, and a second projection extending outward from a second wall of the second body, wherein the first projection is configured to form a hinged connection with the first lip such that the second housing is movable relative to the first housing about the hinged connection, and the second projection is configured to form a snap fit connection with the second lip such that the second housing is immovable relative to the first housing when the second projection and the second lip form the snap fit connection, wherein the first housing and the second housing is configured to apply a clamping force along a perimeter of the pressure relief device to fluidly seal the pressure relief device between the first housing and the second housing when the second projection and the second lip form the snap fit connection.

Any of the pressure relief mechanisms disclosed herein may include any of the following features. The pressure relief mechanism is disposed within a medical device, and is in fluid communication with a pressurized fluid source of the medical device and an outlet of the medical device. The pressure relief mechanism is configured to receive a pressurized fluid from the pressurized fluid source, and move the pressurized fluid to the outlet when the pressurized fluid is below a pressure threshold. The pressure relief device is configured to rupture, and the pressure relief mechanism is configured to divert the pressurized fluid received from the pressurized fluid source away from the outlet and through the pressure relief device, when the pressurized fluid exceeds the pressure threshold.

According to another example, a method for fluidly sealing a pressure relief device within a pressure relief mechanism including a first housing and a second housing, may include coupling a first lip of the first housing to a first projection of the second housing, thereby forming a hinged connection such that the second housing is movable relative to the first housing, moving the second housing relative to the first housing about the hinged connection, coupling a second lip of the first housing to a second projection of the second housing, thereby forming a snap fit connection such that the second housing is immovable relative to the first housing, and fluidly sealing the pressure relief device between the first housing and the second housing by applying a clamping force about the pressure relief device from the first housing and the second housing upon forming the snap fit connection.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The terms “top,” “up,” or “upper” refer to a direction or side of a device relative to its orientation during use, and the terms “bottom,” “down,” or “lower” refer to a direction or side of a device relative to its orientation during use that is opposite of the “top,” “up,” or “upper.” The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device according to some embodiments.

FIG. 2 shows a perspective view of an exemplary pressure relief mechanism according to some embodiments.

FIG. 3 shows a perspective view of a first housing of the pressure relief mechanism of FIG. 2 according to some embodiments.

FIG. 4 shows a top view of the pressure relief mechanism of FIG. 2 according to some embodiments.

FIG. 5 show an exploded perspective view of the pressure relief mechanism of FIG. 2 according to some embodiments.

FIG. 6 shows an exploded perspective view of the pressure relief mechanism of FIG. 2 according to some embodiments.

FIG. 7 shows an exploded perspective view of the pressure relief mechanism of FIG. 2 according to some embodiments.

FIG. 8A shows a perspective view of the pressure relief mechanism of FIG. 2 including a first and second housing in a first position.

FIG. 8B shows a perspective view of the pressure relief mechanism of FIG. 2 with the first and second housing in a second position.

FIG. 8C shows a perspective view of the pressure relief mechanism of FIG. 2 with the first and second housing in a third position.

FIG. 9A shows a cross-sectional side view of the pressure relief mechanism of FIG. 2 with the first and second housing in the second position.

FIG. 9B shows a cross-sectional side view of the pressure relief mechanism of FIG. 2 with the first and second housing in the third position.

### DETAILED DESCRIPTION

Embodiments of this disclosure relate to pressure relief mechanisms incorporated into dispensing devices used to deliver various agents (e.g., powdered agents) to treatment sites within patients during certain medical procedures. For example, dispensing devices may be coupled to a pressurized medium source (e.g., a gas canister) from which a pressurized fluid (e.g., a gas) may be released into the dispensing device for interacting with an agent housed within the dispensing device. The pressurized fluid may mix with the agent to facilitate movement of the agent through the dispensing device, and towards an outlet of the device positioned adjacent to a target treatment site within a subject (e.g., a patient).

The pressurized fluid may be released into the dispensing device at an initial (high) pressure, such as, for example, at approximately 850 psi. In some embodiments, a device (e.g., a pressure regulator) incorporated into the dispensing device may be configured to reduce the initial (high) pressure of the fluid to a second (low) pressure, such as, for example, to approximately 35 psi. Lowering the initial (high) pressure received from the medium source may help ensure that the agent is delivered to the target treatment site at a desired flow rate that is suitable for the intended treatment. If the pressure regulator were to fail, though, the dispensing device may experience damage and/or be rendered inoperable for delivering the agent to the target treatment site. Accordingly, dispensing devices of the present disclosure may include a pressure relief mechanism for housing a pressure relief device, such as, for example, a valve or a burst disc, which may be activated when exposed to a threshold pressure. Upon activation, the pressure relief device may expose the pressurized fluid to atmospheric pressure, thereby allowing the pressurized fluid to escape into the atmosphere and depressurize the dispensing device.

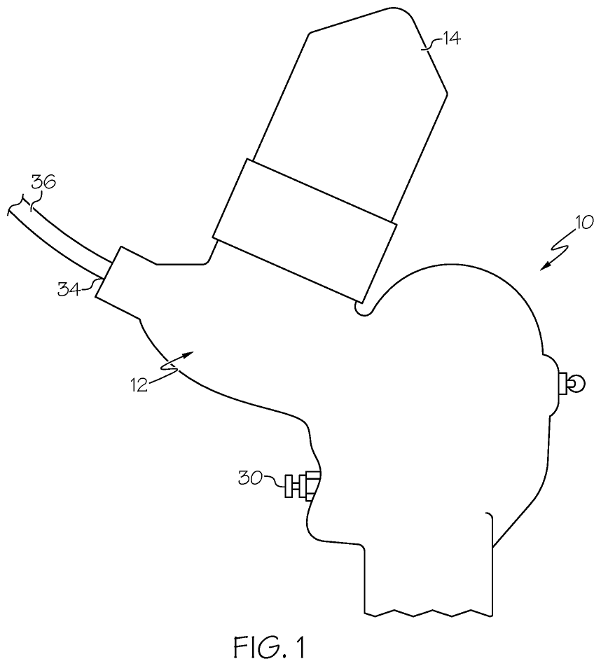

FIG. 1 shows a delivery system 10, which may be an agent (e.g., powder) delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. patent application Ser. No. 16/589,633, filed Oct. 1, 2019, published as U.S. Patent Application Publication No. 2020/0100986 A1 on Apr. 2, 2022, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source in fluid communication with delivery system 10. As described in detail herein, the pressurized fluid released from the pressurized medium source may be received through delivery system 10 at an initial (high) pressure. Actuation mechanism 30 may be selectively actuated (e.g., manually depressible) or otherwise moved or actuated to control delivery of a material (e.g., a powdered agent) and pressurized fluid. The pressurized fluid alone, or a combination of a powdered agent and fluid, may be delivered from an outlet 34 of handle body 12 at a second (low) pressure. Outlet 34 may be in fluid communication with a delivery conduit, for example catheter 36 or another component for delivering the combination of agent and fluid to a desired location within a body lumen of a patient.

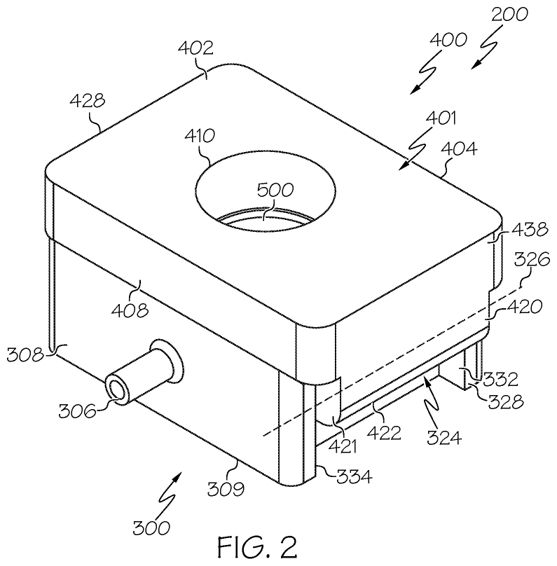

FIG. 2 shows aspects of an exemplary pressure relief mechanism 200, according to some embodiments. Pressure relief mechanism 200 may be housed within handle body 12 of delivery system 10, and in fluid communication with the pressurized medium source. As described herein, pressure relief mechanism 200 may be configured to provide an outlet for the pressurized fluid received through delivery system 10 from the pressurized medium source if the pressure level of the fluid exceeds a threshold value when passing through pressure relief mechanism 200.

In some embodiments, pressure relief mechanism 200 may include a first housing 300, a second housing 400, and a pressure relief device 500. First housing 300 may be configured to engage second housing 400 with pressure relief device 500 disposed therebetween. In some embodiments, first housing 300 may include a base portion of pressure relief mechanism 200, and second housing 400 may include a lid portion of pressure relief mechanism 200. Pressure relief device 500 may include a burst or rupture disc that is configured to elastically deform in response to being exposed to a pressure that exceeds a predetermined threshold value, as described in further detail herein. Each of first housing 300 and second housing 400 may include one or more features or components for securing pressure relief device 500 therebetween when pressure relief mechanism 200 is in a fully assembled state.

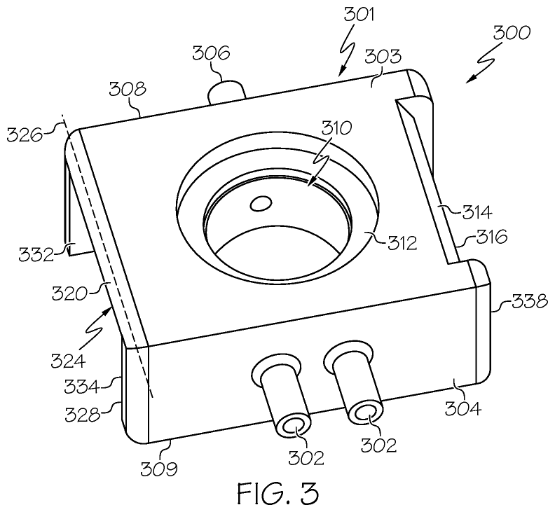

As shown in FIG. 3, for example, first housing 300 may include a body 301 defined by a plurality of walls, such as a first (front) wall 304, a second (rear) wall 308 that is positioned opposite to first (front) wall 304, a first sidewall 328, and a second sidewall 338 that is positioned opposite to first sidewall 328. First housing 300 may include a top (outermost) surface 303, a bottom (outermost) surface 309 that is positioned opposite of top surface 303, a cavity 310 formed along top surface 303, and a shoulder seat 312 disposed within cavity 310. Cavity 310 may be accessible from (e.g., define an opening or recess in) top surface 303 of body 301. Top surface 303 may be approximately perpendicular to first wall 304, second wall 308, first sidewall 328, and second sidewall 338. As described herein, cavity 310 may be configured to receive a pressurized fluid from the pressurized medium source, and one or more devices, such as along shoulder seat 312.

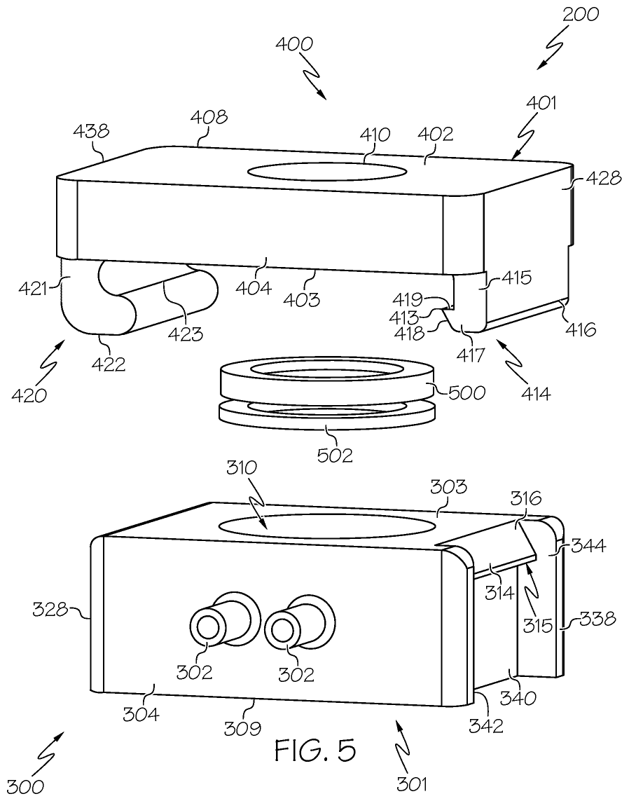

For example, first housing 300 may be configured to receive a gasket 502 and pressure relief device 500 within cavity 310 and along shoulder seat 312 (see FIGS. 5-7). In some embodiments, gasket 502 may be positioned on a top surface of shoulder seat 312 (in the orientation shown in the figures), and pressure relief device 500 may be positioned on a top surface of gasket 502 (in the orientation shown in the figures). In some embodiments, the top surface of gasket 502 may be an outer surface of gasket 502 that is positioned opposite of an opposing outer (bottom) surface of gasket 502, the latter of which may be configured to contact shoulder seat 312.

In this instance, when second housing 400 is coupled to first housing 300, second housing 400 may press against a top surface of pressure relief device 500, and gasket 502 may be compressed between pressure relief device 500 and shoulder seat 312 thereby creating a biasing force between gasket 502 and pressure relief device 500. The biasing force applied to pressure relief device 500 by gasket 502 may cause pressure relief device 500 to be pressed into second housing 400, thereby creating a clamping force around an outer perimeter of pressure relief device 500 for fluidly sealing pressure relief device 500 between first housing 300 and second housing 400.

Referring to FIGS. 2 and 3, first housing 300 may include at least one input port 302 that is disposed through first (front) wall 304, and in fluid communication with cavity 310. In the exemplary embodiment depicted in FIG. 3, first housing 300 includes a pair of input ports 302. First housing 300 may further include at least one output port 306 that is disposed through second (rear) wall 308, and in fluid communication with cavity 310. Input ports 302 may be configured to connect to a source of pressurized fluid (e.g., the pressurized medium source of delivery system 10), and output port 306 may be configured to connect to another component of delivery device 10 (e.g. enclosure 14) to release the pressurized fluid received in cavity 310 via input ports 302. As described herein, pressure relief mechanism 200 may fluidly couple the pressurized fluid source to enclosure 14, and input ports 302 and output port 306 may collectively allow the pressurized fluid to flow through pressure relief mechanism 200.

Although a pair of input ports 302 and a single output port 306 are shown and described herein as being positioned along particular walls of first housing 300, it should be appreciated that first housing 300 may include additional input ports 302 and/or output ports 306 along various other walls without departing from a scope of this disclosure. Alternatively, first housing 300 may include fewer input ports 302 than shown in the drawings without departing from a scope of this disclosure, such as, for example, a single input port 302. In some embodiments, each of the pair of input ports 302 and output port 306 may be configured to connect to a respective connector (e.g., tubing) for receiving and releasing the pressurized fluid, respectively. In some embodiments, input ports 302 and output port 306 may be sized, shaped, and/or otherwise configured to couple to a connector having various suitable dimensions, such as, for example, a diameter of about 5/32 inches.

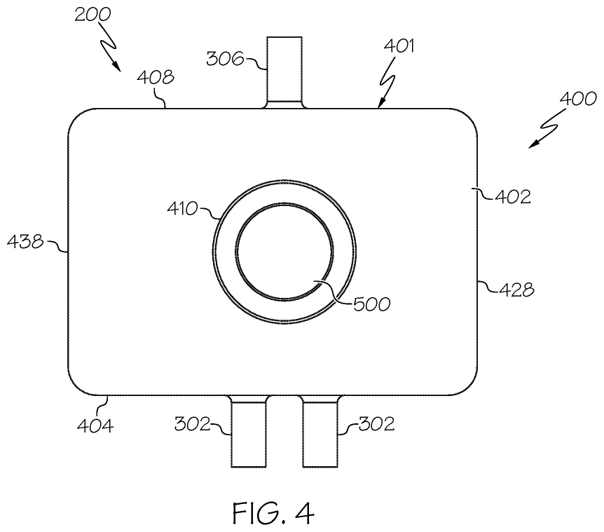

Referring now to FIG. 4, second housing 400 may include a body 401 defined by a plurality of walls, such as a first (front) wall 404, and a second (rear) wall 408 which may be positioned opposite to first (front) wall 404. Body 401 may further include a pair of sidewalls, such as a first sidewall 428 and a second sidewall 438 which may be positioned opposite to first sidewall 428. Body 401 may include a top surface 402 that extends in the same direction as (e.g., substantially parallel to) top surface 303, and top surface 402 may be aligned approximately transverse (e.g., perpendicular) to one or more of first wall 404, second wall 408, first sidewall 428, and second sidewall 438.

Second housing 400 may include an opening 410 extending through body 401, and specifically between a top surface 402 of second housing 400 and an opposite, bottom surface 403 of second housing 400, for example as shown in FIG. 5. Bottom surface 403 may extend in the same direction as (e.g., substantially parallel to) top surface 402, and may be aligned approximately transverse (e.g., perpendicular) to one or more of first wall 404, second wall 408, first sidewall 428, and second sidewall 438. As described in detail herein, opening 410 may be sized, shaped, and/or otherwise configured to align with cavity 310 when first housing 300 and second housing 400 are coupled to one another. In this instance, pressure relief device 500 may be disposed between first housing 300 and second housing 400, and particularly between cavity 310 and opening 410.

As best seen in FIG. 5, body 301 may include a recessed surface 340 extending along second sidewall 338. Recessed surface 340 may be defined by a first interior wall 342 and a second interior wall 344 positioned vertically along (e.g., extending parallel to) second sidewall 338 between top surface 303 and bottom surface 309. First housing 300 may include a first lip 314 extending laterally outward from recessed surface 340 of second sidewall 338. In some embodiments, first lip 314 may extend laterally outward from recessed surface 340 at an angle towards bottom surface 309 of first housing 300.

Second housing 400 may include a first projection 414 and a second projection 420 extending outwardly from body 401, and specifically downward (toward first housing 300) from bottom surface 403 of body 401, for coupling second housing 400 to first housing 300. First projection 414 may be a tapered clip that extends outwardly from first sidewall 428 (e.g., downwardly from bottom surface 403 and radially inward towards second sidewall 438), and second projection 420 may be a rounded clip that extends outwardly from second sidewall 438 (e.g., downwardly from bottom surface 403 and radially inward towards first sidewall 428). In the embodiment, first projection 414 and second projection 420 may be oriented such that an innermost portion of first projection 414, for example, tip 413, and an innermost portion of second projection 420, for example, second portion 423, are opposite of one another. First projection 414 and second projection 420 may be sized, shaped, and/or otherwise configured to interface with a corresponding feature of first housing 300 (e.g., a lip) for coupling second housing 400 thereto.

In some embodiments, first projection 414 may include a first (vertical) portion 415 and a second (lateral) portion 417. First (vertical) portion 415 may extend vertically downward from first sidewall 428 (e.g., such that first portion 415 and first sidewall 428 are approximately co-planar or approximately parallel to one another), and include a distal end 416. Second (lateral) portion 417 may extend laterally inward from distal end 416, towards second projection 420, and may include an upper surface 419 and a tip 413. Second (lateral) portion 417 may define a hook or a ledge, which may include an angled and/or tapered edge 418 for interfacing with a corresponding angled feature of first housing 300 (e.g. first lip 314). In some embodiments, tapered edge 418 may include an angle of approximately 45 degrees relative to a longitudinal length of first (vertical) portion 415.

First projection 414 may be sized, shaped, and/or otherwise configured to interface with first lip 314 when second housing 400 is coupled to first housing 300. For example, an angle of first lip 314 may correspond to an angle of tapered edge 418 to facilitate an engagement between first lip 314 and first projection 414. As described herein, first projection 414 may be configured to form a snap fit connection with first lip 314 when upper surface 419 of second (lateral) portion 417 engages lower interface 315 of first lip 314. Lower interface 315 may be disposed along recessed surface 340 and between top surface 303 and bottom surface 309 of body 301.

Still referring to FIG. 5, second projection 420 may include a first (vertical) portion 421 and a second (lateral) portion 423. First (vertical) portion 421 may extend vertically downward from second sidewall 438 (e.g., such that first portion 421 and second sidewall 438 are approximately co-planar or approximately parallel to one another), and include a distal end 422. Second (lateral) portion 423 may extend laterally inward from distal end 422 and towards first projection 414. In some embodiments, second (lateral) portion 423 may include a bulbous tip, a rounded hook, or a ball joint for interfacing with a corresponding rounded feature of first housing 300 (e.g. a second lip 320). As described herein, second projection 420 may be sized, shaped, and/or otherwise configured to interface with second lip 320 when second housing 400 is coupled to first housing 300. Further, second projection 420 may be configured to allow for rotation of second housing 400 relative to first housing 300 upon engagement of second (lateral) portion 423 with second lip 320.

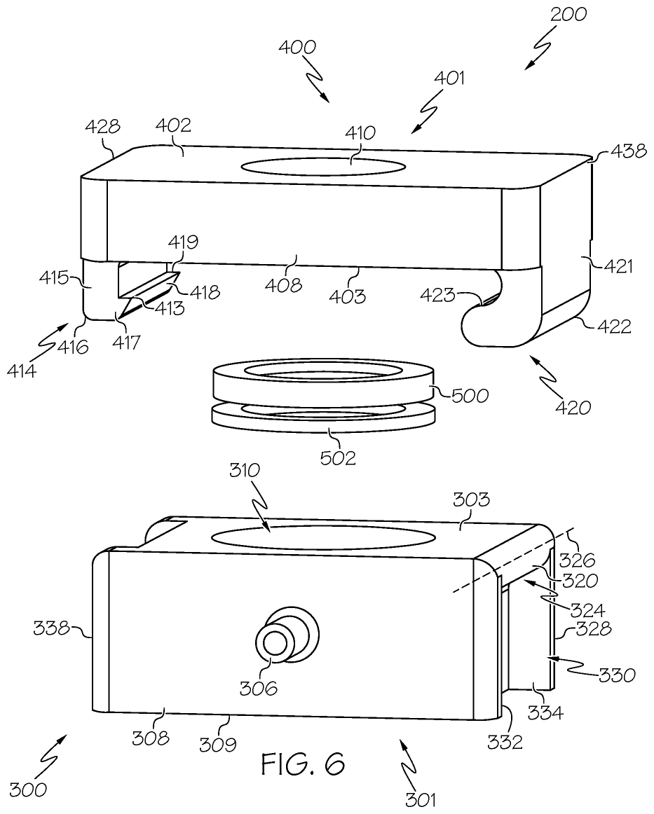

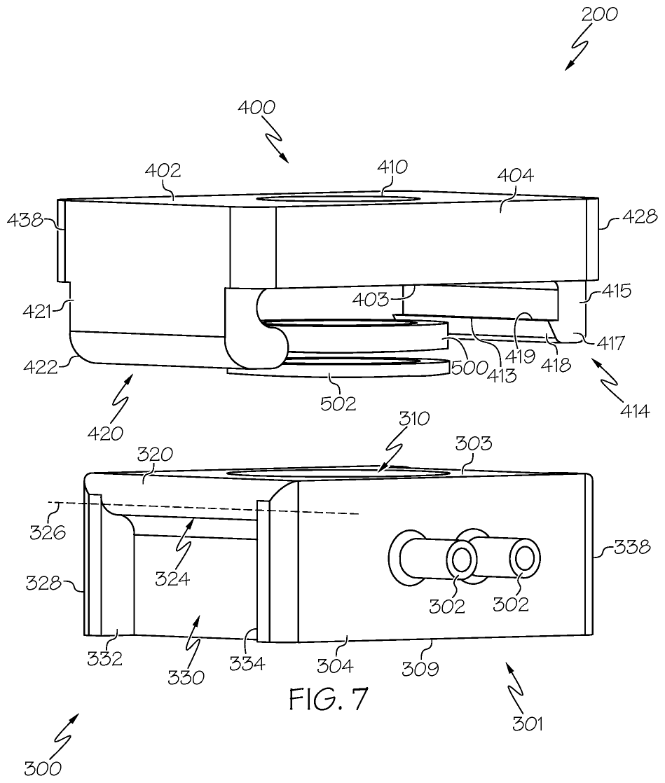

As best seen in FIGS. 6 and 7, body 301 may include a recessed surface 330 extending along first sidewall 328, which may be defined between a first interior wall 332 and a second interior wall 334. In some embodiments, second lip 320 may extend laterally outward from first sidewall 328 at an angle towards bottom surface 309 of first housing 300. Second lip 320 may include a lower interface 324, which may be disposed along recessed surface 330 and between top surface 303 and bottom surface 309 of body 301. A width of second lip 320 may correspond to a lateral distance between first interior wall 332 and second interior wall 334. Lower interface 324 may be sized, shaped, and/or otherwise configured to receive second (lateral) portion 423 when second projection 420 engages second lip 320.

Pressure relief device 500 and gasket 502 may be received between first housing 300 and second housing 400 when pressure relief mechanism 200 is fully assembled. Pressure relief device 500 may be configured to form a fluid seal between cavity 310 and opening 410 such that a pressurized fluid received within cavity 310 (e.g. via input ports 302) are inhibited from exiting pressure relief mechanism 200 via opening 410, and guided toward output port 306. Pressure relief device 500 may be further configured to break, rupture, and/or deform upon cavity 310 receiving the pressurized fluid at a pressure level that exceeds a threshold pressure value. In this instance, pressure relief device 500 may rupture to divert the pressurized fluid out of pressure relief mechanism 200 via opening 410.

In some embodiments, pressure relief device 500 may be a rupture disc formed of a metal film. Pressure relief device 500 may be configured to rupture when exposed to a fluid at a predetermined pressure level that exceeds the threshold pressure value. Once ruptured, the pressurized fluid received within pressure relief mechanism 200 may exit cavity 310 through the ruptured pressure relief device 500 to relieve the high pressure received within cavity 310. Stated differently, to prevent the pressurized fluid from flowing through pressure relief mechanism 200 and toward the other components of delivery system 10 (e.g. enclosure 14, catheter 36, etc.) at a pressure level that exceeds the threshold pressure value, pressure relief device 500 may be configured to rupture and divert the pressurized fluid away from said components. Accordingly, pressure relief mechanism 200 may vent the pressurized fluid into an interior portion of handle body 12, via opening 410.

As described herein, pressure relief mechanism 200 may be configured to generate a clamping force around an outer perimeter of pressure relief device 500 when first housing 300 is coupled to second housing 400. In this instance, pressure relief mechanism 200 may be configured to create an airtight seal between first housing 300 and second housing 400 with pressure relief device 500 disposed therebetween.

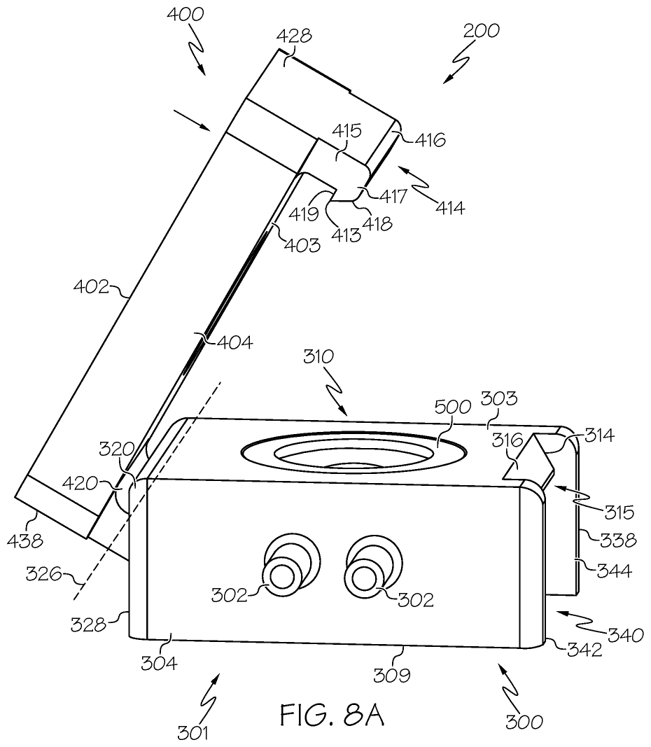

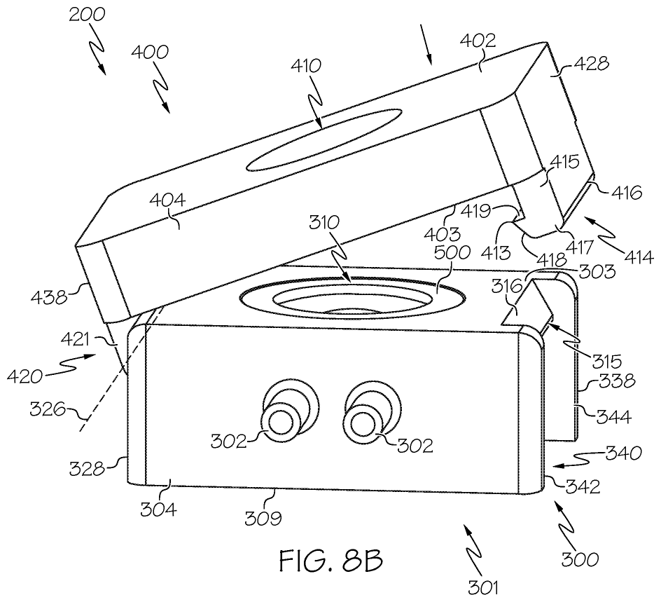

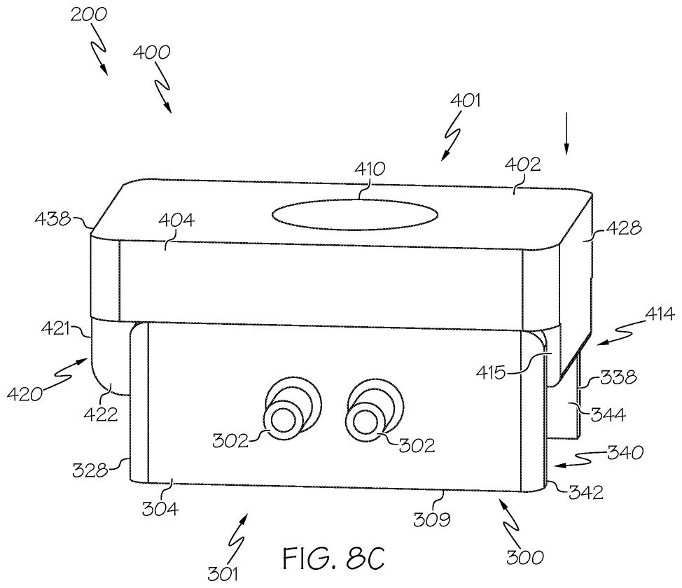

FIGS. 8A-8C depict an exemplary assembly of pressure relief mechanism 200, according to some embodiments. As shown in FIG. 8A, for example, gasket 502 and pressure relief device 500 may be positioned within cavity 310, and particularly along shoulder seat 312. Second lip 320 may be configured to engage second projection 420 to movably couple first housing 300 to second housing 400. In some embodiments, lower interface 324 may form a pocket beneath second lip 320 for receiving at least a portion of second projection 420 to form a hinged connection between first housing 300 and second housing 400. For example, second (lateral) portion 423 defines a hook of second projection 420 that may be sized and shaped to fit into the pocket formed by lower interface 324. In this instance, second projection 420 may be configured to move (e.g., pivot, rotate, etc.) within the pocket and about an axis 326 extending through lower interface 324. In some embodiments, a width of second projection 420 may correspond to the lateral distance between first interior wall 332 and second interior wall 334 such that second projection 420 may fit between and contact each of first interior wall 332 and second interior wall 334. Accordingly, first interior wall 332 and second interior wall 334 may inhibit lateral movement of second projection 420 relative to second lip 320 during assembly and use of pressure relief mechanism 200.

As shown in FIGS. 8B and 8C, second projection 420 may be configured to move (e.g. pivot, rotate, etc.) about axis 326 to move second housing 400 relative to first housing 300, such as between a first position (FIG. 8A), a second position (FIG. 8B), and a third position (FIG. 8C). In some embodiments, second housing 400 and first housing 300 may be arranged in a clamshell configuration such that second housing 400 is in an open configuration when in the first and second positions, and is in a closed configuration when in the third position.

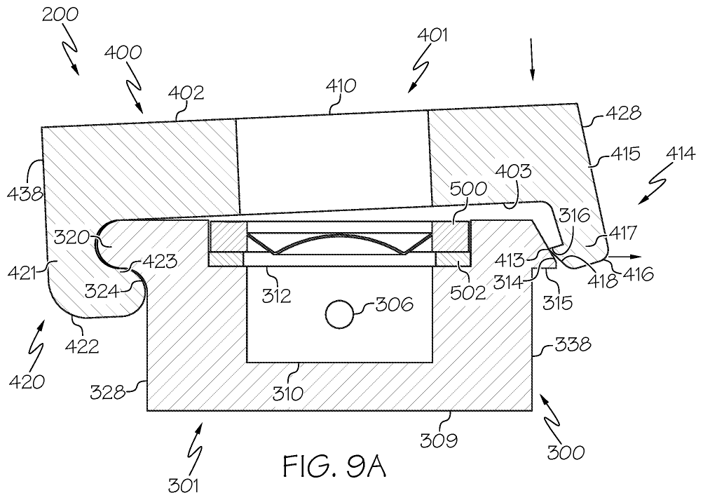

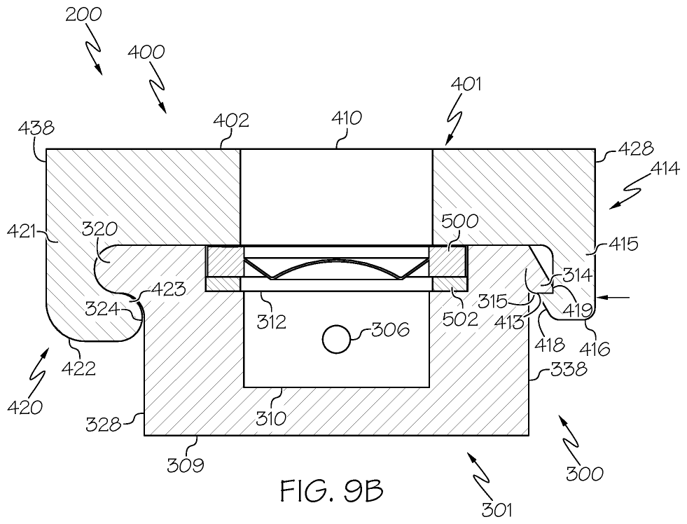

Referring to FIG. 8B, with second projection 420 coupled and movable relative to second lip 320, first projection 414 may be configured to move toward first lip 314. In some embodiments, as shown in FIG. 8C, pressure relief mechanism 200 may be configured to form a snap fit connection when coupling second housing 400 to first housing 300, and specifically between first lip 314 and first projection 414 by moving second sidewall 438 of second housing 400 downward toward first sidewall 328 of first housing 300. It should be appreciated that first projection 414 may encounter first lip 314, and specifically tapered edge 418 may interface with outer surface 316, as best seen in FIG. 9A. In this instance, tapered edge 418 may abut against outer surface 316, and first projection 414 may flex and/or bend laterally outward relative to first sidewall 428 (e.g., away from recessed surface 330) upon contacting first lip 314. First projection 414 may bend laterally outward until upper surface 419 is received within lower interface 315, thereby moving first projection 414 laterally inward relative to first sidewall 428 (e.g., towards recessed surface 330) and securely coupling first projection 414 to first lip 314.

Still referring to FIG. 9A, as second housing 400 is moved toward the third position, it should be appreciated that tapered edge 418 of second (lateral) portion 417 may slide down along outer surface 316 of first lip 314. First projection 414 may be configured to flexibly deform, such as laterally outward relative to first sidewall 428, while abutting against outer surface 316. In other words, first projection 414 may be configured to bend laterally outward relative to first sidewall 428 as first housing 300 and second housing 400 are coupled to one other. Once tip 413 of second (lateral) portion 417 is positioned adjacent to lower interface 315 of first lip 314, second (lateral) portion 417 may return to its original, unbent position, with upper surface 419 of second (lateral) portion 417 engaged against lower interface 315 to form a snap fit connection. The snap fit connection between first projection 414 and first lip 314 may fixedly couple second housing 400 to first housing 300 such that second housing 400 is immovable relative to first housing 300.

The formation of the snap fit connection between first projection 414 and first lip 314 may produce a feedback (e.g. tactile, audible, visual, etc.). For example, when the snap fit connection is formed, an audible sound, such as a click, may be generated to alert a user of the connection between first housing 300 and second housing 400. Alternatively and/or additionally, the snap fit connection may produce tactile or visual feedback such that a user may feel or visualize when pressure relief mechanism 200 is fully assembled.

Referring now to FIG. 9B, with second housing 400 coupled to first housing 300, bottom surface 403 of second housing 400 may press against a surface of pressure relief device 500, which may be positioned on top of gasket 502. A downward force applied by second housing 400 onto pressure relief device 500 may compress gasket 502 against shoulder seat 312, thereby creating a biasing force between gasket 502 and pressure relief device 500. An upward force applied to pressure relief device 500 by gasket 502 may cause pressure relief device 500 to be pressed upward against second housing 400, which may provide a clamping force around an outer perimeter of pressure relief device 500, such as, for example, a circumference of pressure relief device 500 when pressure relief device 500 has a generally circular shape.

In this instance, by providing the clamping force around the outer perimeter of pressure relief device 500, pressure relief mechanism 200 may be configured to fluidly seal pressure relief device 500 between second housing 400 and first housing 300. The snap fit connection between first projection 414 and first lip 314 may ensure that that the clamping force around the outer perimeter of pressure relief device 500 is maintained throughout use of pressure relief mechanism 200. Although pressure relief device 500 is shown as having a generally circular shape or geometry, it should be understood that pressure relief device 500 may have various other suitable shapes and/or configurations without departing from a scope of this disclosure.

As the snap fit connection formed by pressure relief mechanism 200 may be achieved by squeezing second housing 400 and first housing 300 together to generate the clamping force about pressure relief device 500, no tools or fasteners are necessary to assemble pressure relief mechanism 200. Rather, a user may assemble pressure relief mechanism 200 using their hands. Furthermore, first housing 300 and second housing 400 may be made entirely from a moldable polymeric material (e.g. plastic), for example a polycarbonate.

With pressure relief device 500 secured between first housing 300 and second housing 400, one or more connectors (e.g. tubing) may be coupled to pressure relief mechanism 200 via input ports 302 and output port 306 to fluidly couple pressure relief mechanism 200 to a pressurized fluid source and enclosure 14. In this instance, pressure relief mechanism 200 may be configured to control delivery of a pressurized fluid from the pressurized fluid source to enclosure 14 and/or catheter 36 based on a pressurization level of the pressurized fluid received in cavity 310.

As described in detail above, pressure relief mechanism 200 may be configured to guide the pressurized fluid received via input ports 302 through cavity 310 and toward enclosure 14 and/or catheter 36 via output port 306 when the pressurization level of the fluid received therein is at or below a threshold pressure value (e.g. a first (low) pressure). Pressure relief mechanism 200 may be further configured to divert the pressurized fluid received in cavity 310 away from output port 306 in response to pressure relief device 500 rupturing when the pressurization level of the fluid exceeds the threshold pressure value (e.g. a second (high) pressure). In this instance, pressure relief mechanism 200 may form a vent through pressure relief device 500, and fluidly couple cavity 310 to atmospheric pressure within handle body 12 via opening 410. In this instance, pressure relief mechanism 200 may be configured to inhibit the pressurized fluid from moving toward enclosure 14 and/or catheter 36, and divert the pressurized fluid into handle body 12. In other embodiments, pressure relief mechanism 200 may be configured to divert the pressurized fluid to various other suitable locations of delivery device 10 and/or external to delivery device 10.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A pressure relief mechanism, comprising:
a first housing including:
a first body defined between a first wall and a second wall that is opposite of the first wall;
a cavity disposed within the first body between the first wall and the second wall, the first body being configured to receive a pressure relief device within the cavity;
a first lip extending outward from the first wall; and
a second lip extending outward from the second wall; and
a second housing including:
a second body defined between a third wall and a fourth wall that is opposite of the third wall;
a first projection extending outwardly from the third wall; and
a second projection extending outwardly from the fourth wall;
wherein the first projection is configured to engage the first lip and the second projection is configured to engage the second lip to couple the first housing to the second housing with the pressure relief device enclosed within the cavity, wherein the first housing and the second housing are configured to apply a clamping force along a perimeter of the pressure relief device to fluidly seal the pressure relief device between the first housing and the second housing.

2. The pressure relief mechanism of claim 1, wherein the first housing includes an input port disposed through a front wall of the first body, and an output port disposed through a rear wall of the first body;
wherein each of the input port and the output port are in fluid communication with the cavity.

3. The pressure relief mechanism of claim 2, wherein the first housing is configured to receive a pressurized fluid within the cavity via the input port, and release the pressurized fluid from within the cavity via the output port.

4. The pressure relief mechanism of claim 1, wherein the first projection includes a tapered clip configured to form a snap fit connection with the first lip, such that the second housing is immovable relative to the first housing when the tapered clip is coupled to the first lip.

5. The pressure relief mechanism of claim 4, wherein the tapered clip is elastically deformable, and configured to bend laterally outward relative to the third wall as the second housing is coupled to the first housing to form the snap fit connection.

6. The pressure relief mechanism of claim 4, wherein the tapered clip of the first projection includes a hook that extends from the third wall towards the fourth wall, and wherein the hook is configured to engage a lower interface of the first lip.

7. The pressure relief mechanism of claim 4, wherein the second projection includes a rounded clip configured to form a hinged connection with the second lip, such that the second housing is movable relative to the first housing about the hinged connection when the rounded clip is coupled to the second lip.

8. The pressure relief mechanism of claim 7, wherein the rounded clip of the second projection includes a ball joint that extends from the fourth wall towards the third wall, and wherein the ball joint is configured to pivot about an axis when received along a lower interface of the second lip.

9. The pressure relief mechanism of claim 8, wherein the second housing is configured to move between a first position and a second position relative to the first housing about the axis.

10. The pressure relief mechanism of claim 9, wherein, in the first position, the first projection is disengaged from the lower interface of the first lip, and in the second position, the first projection is engaged with the lower interface of the first lip.

11. The pressure relief mechanism of claim 1, wherein the first housing includes at least one recessed surface along the second wall, the at least one recessed surface forming a first interior wall and a second interior wall positioned opposite of the first interior wall.

12. The pressure relief mechanism of claim 11, wherein the second projection of the second housing has a width that corresponds to a distance between the first interior wall and the second interior wall, such that the second projection is configured to fit between the first interior wall and the second interior wall when the second projection engages the second lip.

13. The pressure relief mechanism of claim 12, wherein the first interior wall and the second interior wall are configured to inhibit lateral movement of the second projection relative to the second lip when the first housing is coupled to the second housing.

14. The pressure relief mechanism of claim 1, wherein the second housing includes an opening extending through the second body, and is aligned with the cavity when the first housing is coupled to the second housing.

15. The pressure relief mechanism of claim 14, wherein the opening is configured to release a pressurized fluid received in the cavity of the first housing upon the pressure relief device rupturing.

16. A pressure relief mechanism, comprising:
a first housing including:
a first body;
a cavity disposed within the first body, and configured to receive a pressure relief device;
a first lip extending outward from a first wall of the first body; and
a second lip extending outward from a second wall of the first body; and
a second housing including:
a second body;
a first projection extending outward from a first wall of the second body; and
a second projection extending outward from a second wall of the second body;
wherein the first projection is configured to form a hinged connection with the first lip such that the second housing is movable relative to the first housing about the hinged connection, and the second projection is configured to form a snap fit connection with the second lip such that the second housing is immovable relative to the first housing when the second projection and the second lip form the snap fit connection;
wherein the first housing and the second housing is configured to apply a clamping force along a perimeter of the pressure relief device to fluidly seal the pressure relief device between the first housing and the second housing when the second projection and the second lip form the snap fit connection.

17. The pressure relief mechanism of claim 16, wherein the pressure relief mechanism is disposed within a medical device, and in fluid communication with a pressurized fluid source of the medical device and an outlet of the medical device.

18. The pressure relief mechanism of claim 17, wherein the pressure relief mechanism is configured to receive a pressurized fluid from the pressurized fluid source, and move the pressurized fluid to the outlet when the pressurized fluid is below a pressure threshold.

19. The pressure relief mechanism of claim 18, wherein the pressure relief device is configured to rupture, and the pressure relief mechanism is configured to divert the pressurized fluid received from the pressurized fluid source away from the outlet and through the pressure relief device, when the pressurized fluid exceeds the pressure threshold.

20. A method for fluidly sealing a pressure relief device within a pressure relief mechanism, the pressure relief mechanism including a first housing and a second housing, the method comprising:
coupling a first lip of the first housing to a first projection of the second housing, thereby forming a hinged connection such that the second housing is movable relative to the first housing;
moving the second housing relative to the first housing about the hinged connection;
coupling a second lip of the first housing to a second projection of the second housing, thereby forming a snap fit connection such that the second housing is immovable relative to the first housing; and
fluidly sealing the pressure relief device between the first housing and the second housing by applying a clamping force about the pressure relief device from the first housing and the second housing upon forming the snap fit connection.
